(19) United States
(12) Patent Application Publication
(10) Pub. No.: US 2023/0119138 A1
(43) Pub. Date: Apr. 20, 2023

(54) Oral concentrate additive masking mixute

(71) Applicants: Abraham Souder, Colorado Springs, CO (US); Natalia Goncharova-Souder, Colorado Springs, CO (US); John Steven Souder, Colorado Springs, CO (US)

(72) Inventors: Abraham Souder, Colorado Springs, CO (US); Natalia Goncharova-Souder, Colorado Springs, CO (US); John Steven Souder, Colorado Springs, CO (US)

(21) Appl. No.: 17/566,660

(22) Filed: Mar. 11, 2022

Related U.S. Application Data
(60) Provisional application No. 63/360,581, filed on Oct. 18, 2021.

(51) Int. Cl. A61L 9/012; A23L 29/00

(52) U.S. Cl. CPC A61L 9/012; A23L 29/00

(57) ABSTRACT

The oral concentrated additive Mixture will mask any odor being expelled from the mouth. The concentrate will be used in, but not limited to, a liquid form to be placed on the tongue. It also will be a concentrate to be added to any drinkable liquid to include water, any water based products as well as any soft drink. It also will be added to manufactured candy products as well as any chew-able products to included chewing gum as well as gummy products.

The proprietary ingredients will be stickily tailored to the manufacturer of the concentrate.

A safe ingestible liquid concentrate that can be used (but not limited to) by itself or added/mixed into products to eliminate and/or mask the human scent/odor being expelled from the mouth. This product will mainly be used by, but not limited to, hunters and outdoor enthusiasts.

A couple of drops of this concentrate may be applied to the top of the tongue to be absorbed, which will eliminate the breath odor. Or, can be added to things in conjunction. Such as gum, toothpaste, mouth wash, lozenges, all chewable, edible, products. This concentrate will be safe for all tobacco products as well as jerky products.

This concentrate can also be added to water to make a mouthwash.

1. An Oral Concentrate Additive Masking Mixture is a safe liquid concentrate for the mouth, which is comprising of a liquid that can he added to mouthwash, gum, and can be applied on jerky and tobacco products to mask expelled odor; this specific product helps hide the scent for both hunters and outdoor enthusiasts; It is comprised of hundred percent natural, safe to ingest, biodegradable, environmentally safe and while keeping the flavor of the products; it can be used by itself or mixed with other food, gum, mouthwash, jerky, tobacco products, all chewable and candies, to include but not limited to gummy candy product and lozenges; this product can have added coloring but will not add any odor to the masking additive.
